(19) United States
(12) Patent Application Publication
(10) Pub. No.: US 2024/0100113 A1
(43) Pub. Date: Mar. 28, 2024

(54) TERPENE-CONTAINING COMPOSITIONS

(71) Applicant: QSI HOLDING COMPANY, INC, Pleasant Grove, UT (US)

(72) Inventor: Ann-Celeste Billings, Orem, UT (US)

(21) Appl. No.: 17/951,917

(22) Filed: Sep. 23, 2022

(51) Int. Cl. A61K 36/324; A61K 9/00; A61K 31/015; A61K 31/045; A61K 31/197; A61K 31/198; A61K 31/522; A61K 36/185; A61K 36/28; A61K 36/41; A61K 36/575; A61K 36/68; A61K 36/74; A61K 36/81

(52) U.S. Cl. CPC A61K 36/324; A61K 9/006; A61K 31/015; A61K 31/045; A61K 31/197; A61K 31/198; A61K 31/522; A61K 36/185; A61K 36/28; A61K 36/41; A61K 36/575; A61K 36/68; A61K 36/74; A61K 36/81

(57) ABSTRACT

A composition for oral mucosal delivery can include at least two terpenes having anti-inflammatory properties, the at least two terpenes comprising at least one of terpinolene, beta caryophyllene, alpha pinene, linalool, alpha-terpineol, limonene; and at least two supplements having anti-inflammatory properties, the supplements comprising at least one of cat's claw root extract and boswellia root extract. In other embodiments, the composition can contain other terpenes and active compositions/supplements that have complementary effects.

### BACKGROUND

The present disclosure relates to the terpene-containing compositions that can be administered for the treatment of ailments and as supplements.

### SUMMARY OF DISCLOSURE

According to the present disclosure, a composition can include: from 60% to 70% by weight water; from 10% to 20% by weight flavoring; from 5% to 10% by weight emulsifier; from 3% to 9% by weight sweetener; from 0.2% to 1% by weight a terpene blend, the terpene blend comprising at least two terpenes having anti-inflammatory properties selected from the group consisting of terpinolene, beta caryophyllene, alpha pinene, linalool, alpha-terpineol, limonene, and combinations thereof, from 3% to 6% by weight of a supplement having anti-inflammatory properties, the supplement selected from the group of cat's claw root extract, boswellia root extract, and combinations thereof.

According to one embodiment, the flavoring comprises citric acid. In one embodiment, the composition comprises from 13% to 14% by weight citric acid.

According to one embodiment, the emulsifier comprises 8% by weight lecithin.

According to one embodiment, the supplement comprises 3% to 3.5% by weight cat's claw root extract and 2.5% to 3% by weight of boswellia root extract.

According to one embodiment, the terpene blend comprises 0.6% to 0.7% by weight of a blend of terpinolene, beta caryophyllene, alpha pinene, linalool, alpha-terpineol, and limonene.

According to one embodiment, the sweetener comprises a blend of 3% to 5% glycerin and 1% to 3% by weight stevia leaf extract.

According to one embodiment, a composition for oral mucosal delivery comprises: at least two terpenes having anti-inflammatory properties, the at least two terpenes comprising at least one of terpinolene, beta caryophyllene, alpha pinene, linalool, alpha-terpineol, limonene; and at least two supplements having anti-inflammatory properties, the supplements comprising at least one of cat's claw root extract and boswellia root extract.

According to one embodiment, a composition for oral mucosal delivery comprises: from 50% to 60% by weight water; from 10% to 20% by weight flavoring; from 5% to 10% by weight emulsifier; from 7% to 15% by weight sweetener; from 0.2% to 1% by weight a terpene blend, the terpene blend comprising at least two terpenes having anti-anxiety properties selected from the group consisting of alpha pinene, limonene, linalool, alpha-terpineol, beta caryophyllene, and combinations thereof, from 6% to 12% by weight of a supplement having anti-anxiety properties, the supplement selected from the group of ashwagandha root extract, bacopa leaf extract, caffeine, and combinations thereof.

According to one embodiment, the emulsifier aids liposomal delivery of the supplement, and the emulsifier comprises 8% by weight lecithin.

According to one embodiment, the supplement comprises 5% to 6% by weight caffeine, 3% to 3.5% by weight ashwagandha root extract, and 2.5% to 3% by weight of bacopa leaf extract.

According to one embodiment, the terpene blend comprises 0.6% to 0.7% by weight of a blend of terpinolene, beta caryophyllene, alpha pinene, linalool, alpha-terpineol, and limonene.

According to one embodiment, the sweetener comprises a blend of 3% to 5% glycerin and 1% to 3% by weight stevia leaf extract.

According to one embodiment, a composition for oral mucosal delivery comprises: from 60% to 70% by weight water; from 10% to 20% by weight flavoring; from 6% to 11% by weight emulsifier; from 5% to 10% by weight sweetener; from 0.2% to 1% by weight a terpene blend, the terpene blend comprising at least two terpenes having anti-anxiety properties selected from the group consisting of limonene, terpinolene, linalool, alpha-terpineol, beta caryophyllene and combinations thereof, from 5% to 10% by weight of a supplement having anti-anxiety properties, the supplement selected from the group of magnolia bark extract, passionflower extract, chamomile flower extract, and combinations thereof.

According to one embodiment, the supplement comprises 4% to 6% by weight magnolia bark extract, 1% to 3% by weight passionflower extract, and 0.5% to 1.5% by weight of chamomile flower extract.

According to one embodiment, the terpene blend comprises 0.6% to 0.7% by weight of a blend of limonene, terpinolene, linalool, alpha-terpineol, and beta caryophyllene.

According to one embodiment, the sweetener comprises a blend of 3% to 5% glycerin and 1% to 3% by weight stevia leaf extract.

According to one embodiment, a composition for oral mucosal delivery comprises: from 65% to 75% by weight water; from 1% to 10% by weight flavoring; from 6% to 12% by weight emulsifier; from 5% to 10% by weight sweetener; from 0.2% to 1% by weight a terpene blend, the terpene blend comprising at least two terpenes having anti-anxiety properties selected from the group consisting of beta caryophyllene, linalool, alpha-terpineol, limonene, and combinations thereof; from 5% to 10% by weight of a supplement having anti-anxiety properties, the supplement selected from the group of GABA, rhodiola root, L-Theanine, and combinations thereof.

According to one embodiment, the supplement comprises 4% to 5% by weight GABA, 3% to 4% by weight Rhodiola rosea, and 0.5% to 1.5% by weight L-theanine.

According to one embodiment, the terpene blend comprises 0.5% to 0.7% by weight of a blend of beta caryophyllene, linalool, alpha-terpineol, and limonene.

### DETAILED DESCRIPTION

The following provides a detailed description of particular embodiments of the present disclosure. The description is only exemplary of the principles of the present invention and should not be viewed as narrowing the scope of the claims which follow, which claims define the full scope of the invention. The described features, structures, or characteristics of configurations of the invention may be combined in any suitable manner in one or more configurations. Such combinations are specifically contemplated as being within the scope of the present invention, regardless of whether they are explicitly described as a combination herein. In some cases, detailed descriptions of well-known items or repeated descriptions of substantially the same configurations may be omitted. This facilitates the understanding of those skilled in the art by avoiding an unnecessarily redundant description. All statements herein reciting principles, aspects, and embodiments of the invention, as well as specific examples thereof, are intended to encompass equivalents thereof.

As used herein, the term “percent weight” refers to the amount of a component relative to the entire weight of the composition, expressed as:

(mass of the component/mass of the composition)×100%.

Unless stated to the contrary, implicit from the context, or customary in the art, all parts and percents are based on weight and all test methods are current as of the filing date of this disclosure. It is understood that the sum of the components in each of the mixtures and compositions, disclosed herein yields 100 weight percent.

In some examples, the composition can be delivered orally, mucosally, cutaneously, transdermally, ocularly, transfollicularly, nasally, percutaneously, etc. In one example, the composition is delivered under the user's tongue via oral mucosal delivery. Because the oral mucosa is highly vascularized, drugs that are absorbed through the oral mucosa directly enter the systemic circulation, bypassing the gastrointestinal tract and first-pass metabolism in the liver. For some drugs, this results in rapid onset of action via a more comfortable and convenient delivery route than alternatives.

Some embodiments disclosed herein comprise a composition for oral delivery that includes a therapeutically effective amount of an active composition or supplement as well as a terpene that compliments the therapeutic effective of the active composition or supplement. That is, the terpene can also have a similar therapeutic effect as the active composition. In other configurations, the terpene can have a different therapeutic effect compared to the active composition or supplement.

The present disclosure provides a composition. The composition generally contains (A) an active composition or supplement, (B) a blend of terpenes, and (C) an emulsifier. The composition also optionally includes (D) a preservative, (E) flavoring (which can include a sweetener), and/or (F) a liquid carrier.

A. Active Composition or Supplement

As used herein, “active composition” means any of the following: relaxants (L-theanine, rhodiola root, GABA, magnolia bark extract, passionflower extract, chamomile flower extract, valerian root, 5-HTP, lemon balm, schisandra, Rhodiola rosea, holy basil, magnesium, Chaga mushroom, Melatonin), anti-inflammatories (cat's claw root extract, boswellia root extract, ginger, white willow bark, turmeric, garlic, curcumin), nootropics (natural or synthetic caffeine, L-theanine, creatine, Bacopa monnieri, Rhodiola rosea, ginseng, Ginkgo biloba, Noopept, piracetam, phenylpiracetam, green coffee bean extract, green tea extract, green tea polyphenols, choline, acetylcholine, Lion's Mane mushroom, alpha GPC, NAC, epicatechin, piperine, artichoke extract, forskolin, agmatine, L-phenylalanine, DL-phenylalanine, cocoa), and anti-anxiety/anti-stress compositions (ashwagandha root extract, bacopa leaf extract, L-theanine, rhodiola root). An active composition that has relaxation properties is a chemical or compound known to reduce stress and/or anxiety. An active composition that has anti-inflammatory properties is a chemical or compound known to reduce inflammation.

In an embodiment, the active composition or supplement is mushrooms (such as Reishi, Cordyceps, Lion's Mane, Turkey Tail, Chaga).

In an embodiment, the active composition or supplement is at least one of an active composition known to improve circulation and/or sexual health. In an embodiment, the active composition is at least one of L-citrulline, aspartic acid, yohimbe, maca, phlomis umbrosa, cynanchum wilfordii, black ginger, angelica gigas, jambu (acmella oleracea or spilanthes acmella), and eleuthero (Siberian ginseng).

In an embodiment, the active composition or supplement is an amino acid. Nonlimiting examples of suitable amino acids include tryptophan, arginine, phenylalanine, tyrosine, histidine, arginine, alanine, glycine, serine, threonine, leucine, isoleucine, methionine, valine, theanine, and combinations thereof. In an embodiment, the amino acid is selected from L-tryptophan, L-arginine, L-phenylalanine, L-tyrosine, L-histidine, L-arginine, 5-hydroxytryptophan (5-HTTP), theanine, and combinations thereof.

In an embodiment, the amino acid is 5-hydroxytryptophan (5-HTTP). 5-HTTP is also known as oxitriptan.

In an embodiment, the amino acid is selected from L-tryptophan, L-arginine, L-phenylalanine, L-tyrosine, L-histidine, L-arginine, L-theanine, and combinations thereof.

In an embodiment, the supplement is gamma aminobutyric acid (GABA). GABA is also known as 4-aminobutanoic acid.

In an embodiment, the supplement is caffeine. Caffeine is also known as 1,3,7-trimethylxanthine.

In an embodiment, the supplement is selected from L-tryptophan, L-arginine, L-phenylalanine, L-tyrosine, L-histidine, L-arginine, 5-HTTP, pyridoxine, piracetam, GABA, theobromine, caffeine, resveratrol, and combinations thereof.

In an embodiment, the active composition or supplement is a plant herb. A “plant herb” is a medicinal preparation prepared from a plant or plant part. Nonlimiting examples of suitable plant herbs include rhodiola, cat's claw root extract, boswellia root extract, bacopa leaf extract, magnolia bark extract, ashwagandha, ginseng, passionflower extract, chamomile flower extract, and combinations thereof. The plant herb may be in the form of a powder, an extract, a pulp, a leaf, a stem, a flower, a petal, a seed, a root, or a combination thereof.

In an embodiment, the plant herb is rhodiola. “Rhodiola” is a medicinal preparation from the plant Rhodiola rosea. In an embodiment, the rhodiola contains one or more of phenols, rosavin, rosin, rosarin, organic acids, terpenoids, phenolic acids and their derivatives, flavonoids, anthraquinones, alkaloids, tyrosol, and salidroside.

In an embodiment, the plant herb is cat's claw root extract. “Cat's claw root extract” is a medicinal preparation from the plant cat's claw vine. In an embodiment, cat's claw root extract includes one or more of oxindole alkaloids (isopteropodine, pteropodine, rhynchophylline, mytraphylline, speciophylline, uncarine F, uncarine E), indole alkaloidal glucosides (cadambine, 3-dihydrocadambine, and 3-isodihydrocadambine), quinovic acid glycosides, tannins, polyphenols, catechins and beta sitosterol. In vitro, extracts of cat's claw have immune modulatory, antiviral and antimutagenic activities.

In an embodiment, the plant herb is boswellia root extract. “Boswellia root extract” is a medicinal preparation from the plant Boswellia serrata (family burseracea). In an embodiment, boswellia root extract contains one or more of resin, amino acids, phenols, terpenes, polysaccharides, 11-keto-beta-boswellic acid (AKBA), and β-boswellic acid, the major active anti-inflammatory component. Extract of B. serrata or β-boswellic acid has been reported to inhibit hypersensitivity reactions by regulating both the humoral and cellular immune systems. β-boswellic acid can decrease primary antibody synthesis, inhibit polymorphonuclear leukocyte proliferation and infiltration, enhance the phagocytotic function of macrophages, suppress the classical and alternate complement pathways, and suppress the inflammation process.

In an embodiment, the plant herb is bacopa leaf extract. “Bacopa leaf extract” is a medicinal preparation from the plant Bacopa monnieri (also known as brahmi, water hyssop, Bacopa monniera, and Herpestis monniera). In an embodiment, bacopa leaf extract contains one or more of triterpenoid saponins known as bacosides (with jujubogenin or pseudo-jujubogenin moieties as aglycone units) brahmine, nicotine, herpestine, d-mannitol, apigenin, hersaponin, monnierasides I-III, cucurbitacins and plantainoside B. Bacopa leaf extract exhibits the following mechanisms-anti-oxidant neuroprotection (via redox and enzyme induction), acetylcholinesterase inhibition and/or choline acetyltransferase activation, β-amyloid reduction, increased cerebral blood flow, and neurotransmitter modulation (acetylcholine, 5-hydroxytryptamine, dopamine). Bacopa monnieri demonstrates anti-oxidant, hepatoprotective, and neuroprotective activity.

In an embodiment, the plant herb is magnolia bark extract. “Magnolia bark extract” is a medicinal preparation from the plant Magnolia officinalis (also known as Magnoliae cortex, Magnolia officinalis cortex, and Magnolia officinalis bark). In an embodiment, magnolia bark extract contains one or more of neolignans (such as magnolol (5,5′-diallyl-2,2′-dihydroxybiphenyl), honokiol (5,3′-diallyl-2,4′-dihydroxybiphenyl)), lignans, sesquiterpenes, terpenes, alkaloids (such as benzyltetrahydroisoquinoline, N-methylcoxylonine, (S)-magnoflorine, magnofficine, (R)-asimilobine, corytuberine, anonaine, liriodenine and (2) the benzyltetrahydroisoquinoline alkaloids (R)-magnocurarine, (S)-tembetarine, lotusine, (R)-oblongine, reticuline), and phenylethanoid glycosides.

In an embodiment, the plant herb is passionflower extract. “Passionflower extract” is a medicinal preparation from the plant Passiflora incarnata, also known as Purple Passionflower.

In an embodiment, passionflower extract contains one or more of quercetol, kaempferol, apigenin, luteolin, vitexin, saponaroside, schaftoside, isoschaftoside, isovitexin, isoorientin, sitosterol, stigmasterol, maltol, chrysin, coumerin, and umbelliferon.

In an embodiment, the plant herb is chamomile flower extract. “Chamomile flower extract” is a medicinal preparation from the plant Matricaria recutita L. (also known as amomile, German Chamomile, Roman Chamomile, English Chamomile and Flos Chamomile). In an embodiment, the chamomile extract contains one or more of chamazulene, alpha-bisabolol, bisabolol oxides A, B and C, bisabolone oxide, luteolol, apigenol, quercetol, umbelliferone, herniarin, matricine, and matricarina.

In an embodiment, the plant herb is ashwagandha or ashwagandha root extract. “Ashwagandha” is a medicinal preparation from the plant Withania somnifera. In an embodiment, the ashwagandha contains one or more of sitoindoside, withanolide (withaferin A and/or withanone), acylsterylglucoside, triethylene glycol, alkaloids, withanamides, withanosides, withanolide glycosides, and steroidal saponins.

In an embodiment, the plant herb is ginseng. “Ginseng” is a medicinal preparation from plants of the genus Panax, such as Panax ginseng, Panax notoginseng, Panax qidnquefolius, and combinations thereof. In an embodiment, the ginseng contains one or more of ginsenosides (e.g., Rg1, Rc, Rd, Re, Rb1, Rb2, RbO, etc.) and pseudoginsenosides.

In an embodiment, the supplement is selected from cat's claw root extract, boswellia root extract, and combinations thereof.

In an embodiment, the supplement is selected from caffeine, ashwagandha root extract, bacopa leaf extract, and combinations thereof.

In an embodiment, the supplement is selected from magnolia bark extract, passionflower extract, chamomile flower extract, and combinations thereof.

In an embodiment, the supplement is selected from GABA, rhodiola root, L-Theanine, and combinations thereof.

In some embodiments, the composition contains from 1 wt % to 20 wt %, or from 5 wt % to 15 wt %, or from 6 wt % to 12 wt % of the supplement or active composition, based on the total weight of the composition. In an embodiment, the composition contains 5 wt %, or 6 wt %, or 7 wt %, or 8 wt %, or 9 wt %, or 10 wt %, or 11 wt %, or 12 wt %, or 13 wt %, or 14 wt %, or 15 wt % of the supplement, based on the total weight of the composition. In another embodiment, the composition contains from about 5 wt % to about 12 wt % of the supplement, based on the total weight of the composition.

The active composition may be present in the composition in a therapeutically effective amount. The therapeutically effective amount may be a dosage that is small enough to effectively treat a patient, such as reduce stress, reduce anxiety, increase calm, increase focus, etc. A therapeutically effective amount of an active composition means the amount of a compound that, when administered to a subject for treating a disease, is sufficient to effect such treatment for the disease. The therapeutically effective amount can vary depending on the compound, the disease and its severity, and the age, weight, etc., of the subject to be treated. Where the active composition is a supplement, the therapeutically effective amount can also be an amount of active composition or supplement to provide preventative benefits to a user.

B. Blend of Terpenes

As used herein, “terpene” means any natural or synthetic compound with the formula (C₅H₈)ₙ, as well as any terpenoids (the oxygenated derivatives of terpenes). Terpenes are naturally present in many essential oils. Essential oils are a type of metabolites found in vegetables, and their chemical composition is diverse, with monoterpenes and sesquiterpenes generally being the most abundant molecules. Many of these molecules with 10 and 15 carbon atoms have an especially important biological activity, including functionalities as antimicrobial, antifungal, antioxidant, anti-inflammatory, insecticide, analgesic, anticancer, cytotoxic, among others.

“Terpene” encompasses the following terpenes and terpenoids: Limonene, cineole, fenchone, and citral, Ursolic acid, Limonene, Paeoniflorin, Rebaudioside A, farnesol, Eucaliptol, Menthol, Tripterine, Ursolic acid and anethole, Thymol, Docetaxel, Forskolin, Paeoniflorin, Triptolide, β-citronellene, α-bisabolol, Hyperforin, Cineole, Squalene, Paclitaxel, Madecassoside, Asiaticoside, Triterpenoids of Ganoderma l., Safranal, Ursolic acid, Farnesol, oleanoic acid, Docetaxel, Limoneno, 1,8-cineole, Betulin, Astragaloside IV, Lupane, Hurpezine A, Asiaticoside, Triptolide, Cineole, Sericoside, Madecassoside, asiaticoside, Betulin, Sericoside, α-Terpineol, γ-Terpineol, Borneol, isoborneol, Terpinen-4-ol, Nerol, Lavandulol, Myrcene α-Pinene, β-Pinene, Camphene, (E)-β-Ocimene, (Z)-β-Ocimene, β-Phellandrene, Eucalyptol, β-caryophyllene, β-Farnesene, Germacrene, α-Humulene, andrographolide, curcumin, beta-pinene, alpha-bisabolol, borneol, camphene, camphor, delta-3-carene, caryophyllene oxide, alpha-cedrene, beta-eudesmol, fenchol, geraniol, guaiol, alpha-humulene, isoborneol, limonene menthol, myrcene, neral, nerolidol, cis-ocimene, trans-ocimene, alpha-phellandrene, alpha-pinene, beta-pinene, sabinene, alpha terpinene, alpha-terpineol, terpinolene, alpha-guaiene, elemene, farnesene, farnesol, germacrene B, guaia-1(10), 11-diene, trans-2-pinanol, selina-3,7(11)-diene, eudesm-7(11)-en-4-ol, and valencene.

By way of example and not of limitation, “terpene” as used herein can encompass the following monoterpenes: pinene, myrcene, limonene, eucalyptol, thujene, myrcene, linalool, citral, camphor, menthol, Lavandulol, terpineol, citronellol, etc.

By way of example and not of limitation, “terpene” as used herein can encompass the following sesquiterpenes: bisabolene, zingiberene, germacrene, caryophyllene, chamazulene, caryophyllene oxide, germacrene, artemisinic acid, patchoulene, humulene, bergamotene, farnesene, eudesmol, etc.

By way of example and not of limitation, “terpene” as used herein can encompass the following sesquiterpenes: retinol, taxol and phytol.

By way of example and not of limitation, “terpene” as used herein can encompass the following triterpenes: squalene, sitosterol, and other phytosterols.

By way of example and not of limitation, “terpene” as used herein can encompass the following tetraterpenes: carotenes, lycopene, and bixin.

In an embodiment, the terpene blend is selected from terpinolene, beta caryophyllene, alpha pinene, linalool, alpha-terpineol, limonene, and combinations thereof.

In an embodiment, the terpene blend is selected from alpha pinene, limonene, linalool, alpha-terpineol, beta caryophyllene, and combinations thereof.

In an embodiment, the terpene blend is selected from limonene, terpinolene, linalool, alpha-terpineol, beta caryophyllene, and combinations thereof.

In an embodiment, the terpene blend is selected from beta caryophyllene, linalool, alpha-terpineol, limonene and combinations thereof.

In some embodiments, the composition contains from 0.1 wt % to 2 wt %, or from 0.3 wt % to 1.5 wt %, or from 0.4 wt % to 1 wt % of the terpene blend, based on the total weight of the composition. In an embodiment, the composition contains 0.1 wt %, or 0.2 wt %, or 0.3 wt %, or 0.4 wt %, or 0.5 wt %, or 0.6 wt %, or 0.7 wt %, or 0.8 wt %, or 0.9 wt %, or 1 wt %, or 1.1 wt % of the terpene blend, based on the total weight of the composition. In another embodiment, the composition contains from about 0.4 wt % to about 0.8 wt % of the terpene blend, based on the total weight of the composition.

C. Emulsifier

With respect to the emulsifier, any suitable emulsifier can be used. For example, lecithin or other emulsifiers can be used. The emulsifier can also serve to create a liposomal format for delivery of the active composition. Liposomes are sphere-shaped vesicles consisting of one or more phospholipid bilayers and can be effective for delivery of small molecules. For example, lecithin provides a known method of encapsulation and delivery for small molecules. Liposomal format for delivery can also reduce undesired strong flavors present in some terpenes. Soy derived lecithin, sunflower lecithin, or any other type of lecithin can be used. Hydrocolloids, such as locust bean gum, carrageenan, pectin, guar gum, gellan gum, carrageenan, and starch, can also be used as emulsifiers. Other emulsifiers, such as mono- and diglycerides, polysorbates, etc. can also be used.

D. Preservative

A preservative can be optionally included in the composition. Any known preservatives can be used, and in some examples the composition contains a naturally derived preservative.

In an embodiment, potassium sorbate is the preservative.

In some embodiments, the composition contains from 0.05 wt % to 0.15 wt %, or from 0.08 wt % to 0.13 wt %, or from 0.9 wt % to 0.11 wt % of the preservative, based on the total weight of the composition. In an embodiment, the composition contains 0.1% by weight of the preservative.

E. Flavor

Because the composition may be delivered via oral mucosal delivery, the composition may be a liquid composition, and the composition may also include one or more flavorings to improve the delivery experience.

With respect to the flavoring, any suitable flavoring can be used. In some examples, natural flavorings are used. Any suitable natural or artificial flavoring can be used. For example, citric acid or other fruit extracts can be used (and can have the benefit of reducing bitterness from terpenes). In one embodiment, citric acid comprises up to half of the total amount of flavor additive.

Sweeteners can also be used to improve the oral taste of the composition. For example, stevia, sugar, glycerin, sucralose, etc., can be used to sweeten the composition. The terpenes that are included in the composition can also often provide a flavoring themselves.

F. Liquid Carrier

The remainder of the composition can be a liquid solution. In an embodiment, water is used as the liquid carrier. In another embodiment, a hydrophilic liquid solution is used for the remainder of the composition.

One or more bioavailability enhancers can also be included in the formulation, for example, ascorbic acid, benzyl alcohol, methylparaben, propylparaben, benzalkonium chloride, thiomersal, sodium bisulfite, sodium metabisulfite or thiourea can be used. Bioavailability enhancers can increase the stability of the active composition and/or the ability of the active composition to cross membranes.

In an embodiment, the composition contains about 2% to about 10% of one or more active compositions/supplements, or about 3% to about 9% of one or more active compositions/supplements, or about 4% to about 8% of active compositions/supplements, or about 4% to about 6% of active compositions/supplements, or about 6% active compositions/supplements. The value of the active compositions/supplements can be equal to any integer value or values within any of the above-described numerical ranges, including the end-points of the range.

In an embodiment, the composition can contain about 1% to about 5% uncaria tomentosa, or about 2% to about 4% uncaria tomentosa, or about 3.2% uncaria tomentosa. The remainder of the active composition of the composition can comprise boswellia serrata. For example, the example, the composition can contain about 1% to about 5% Boswellia serrata, or about 2% to about 4% boswellia serrata, or about 2.7% boswellia serrata. The value of the active composition can be equal to any integer value or values within any of the above-described numerical ranges, including the end-points of the range.

The composition can also contain about 0.2% to about 1% of at least two different types of terpenes, about 0.3% to about 0.9% of at least two different types of terpenes, about 0.4 to about 0.8% of at least two different types of terpenes, about 0.5% to about 0.7% of at least two different types of terpenes, or about 0.6% of at least two different types of terpenes. The value of the terpenes can be equal to an integer value or values within any of the above-described numerical ranges, including the end-points of the range. In one example, about 0.65% of at least two types of terpenes are included in the composition. Exemplary terpenes that can have a complementary effect to uncaria tomentosa and/or boswellia serrata (i.e., known anti-inflammatories) include terpinolene, beta caryophyllene, alpha pinene, linalool, alpha-terpineol, limonene. In one configuration, a blend of black pepper, pine oil, lavender, and lemon together comprise about 0.63% of the composition.

The composition can also contain about 0.1% preservative. Any known preservatives can be used, and in some examples the composition contains a naturally derived preservative. Potassium sorbate, etc. can be used.

In an exemplary formulation (Table 1: Comfort Composition), the formulation is comprised of about 66.2% water, 13.3% citric extract, 8% sunflower lecithin, 4% glycerin, 3.2% of the active composition cat's claw root extract (anti-inflammatory), 2.7% of the active composition Boswellia root extract (anti-inflammatory), 2% stevia leaf extract, 0.63% of a terpene blend with anti-inflammatory and/or entourage effects (comprised of terpinolene, beta caryophyllene, alpha pinene, linalool, alpha-terpineol, limonene), and about 0.1% potassium sorbate or another preservative.

TABLE 1
Comfort Composition

| | | | | Quantitative (by | |
|---|---|---|---|---|---|
| Qualitative | | | | Weight (KG, G, | |
| Description | | | | MG, MCG) or | |
| (Common | | | Quantitative | Volume (KL, L, | |
| Name): | Source Name: | Function: | (%): | ML): | |
| Purified Water | Aqua | Water | 66.2% | 662 | mg |
| Natural Flavors | Fruit extracts, | Flavor | 13.3% | 133 | mg |
| | Citric acid | | | | |
| Lecithin | Sunflower | Excipient - | 8.0% | 80 | mg |
| | | Emulsifier | | | |
| Glycerin | Palm kernel | Sweetener | 4.0% | 40 | mg |
| | (Elaeis | | | | |
| | guineensis) | | | | |
| Cat's Claw root | Uncaria | Active- anti- | 3.2% | 32 | mg |
| extract | tomentosa | inflammatory | | | |
| Boswellia root | Boswellia | Active- anti- | 2.7% | 27 | mg |
| extract | serrata | inflammatory | | | |
| Stevia Leaf | Stevia | Sweetener | 2.0% | 20 | mg |
| Extract | rebaudiana | | | | |
| Terpenes- | Black pepper oil | Anti- | 0.63% | 6.3 | mg |
| terpinolene, beta | (Piper nigrum), | inflammatory, | | | |
| caryophyllene, | Pine oil | entourage effect | | | |
| alpha pinene, | (Cupressus | | | | |
| linalool, alpha- | macrocarpa), | | | | |
| terpineol, | Lavender | | | | |
| limonene | (Lavendula | | | | |
| | angustifolia), | | | | |
| | Lemon (limon | | | | |
| | citrus) | | | | |
| Potassium | Synthesis | Preservative | 0.1% | 1 | mg |

Sorbate

In another example, the composition can contain from about 50% to about 60% by weight of water or another hydrophilic liquid, about 10% to about 20% by weight of natural flavors, about 5% to about 10% by weight of emulsifier, about 5% to about 15% by weight of sweetener, about 5% to about 15% of one or more active compositions, about 0.2% to about 1% of terpenes, and about 0.05% to 0.15% of a preservative.

More specifically, the composition can contain from about 50% to about 60% water, or about 5% to about 55% water, or about 52% to about 54% water, or about 53.8% water. The value of the water in the composition can be equal to an integer value or values within any of the above-described numerical ranges, including the end-points of the range.

The composition can contain from about 10% to about 20% by weight natural flavoring (such as fruit extracts, citric acid, etc.), or about 12% to about 18% by weight natural flavoring, or about 14% to about 16% by weight of natural flavoring, or about 15% by weight natural flavoring. The value of the natural flavoring in the composition can be equal to an integer value or values within any of the above-described numerical ranges, including the end-points of the range.

The composition can contain about 5% to about 10% by weight of emulsifier, or about 6% to about 9% by weight of emulsifier, or about 7% to about 8% by weight of emulsifier. The value of the natural flavoring in the composition can be equal to an integer value or values within any of the above-described numerical ranges, including the end-points of the range.

The composition can contain about 5% to about 15% by weight of sweetener, or about 7% to about 13% by weight of sweetener, or about 11% by weight of sweetener. The value of the sweetener in the composition can be equal to an integer value or values within any of the above-described numerical ranges, including the end-points of the range. Additionally, more than one sweetener can be used to achieve this overall amount of sweetener within the composition. For example, the composition can contain about 5% to about 10% by weight of stevia leaf extract, or about 6% to about 8% by weight of stevia leaf extract, or about 7% by weight of stevia leaf extract. The value of the stevia leaf extract in the composition can be equal to an integer value or values within any of the above-described numerical ranges, including the end-points of the range. The composition can also contain about 2% to about 6% by weight of glycerin, or about 3% to about 5% by weight of glycerin, or about 4% by weight of glycerin. The value of the glycerin in the composition can be equal to an integer value or values within any of the above-described numerical ranges, including the end-points of the range. The use of glycerin as a sweetener can also have the added benefit of stabilizing the emulsifier to improve liposomal deliver of active compositions.

The composition can contain about 5% to about 15% of one or more active compositions, such as one or more active compositions known to be nootropic, anti-anxiety, and/or anti-stress. In some examples, the composition can contain about 3% to about 7% natural caffeine, or about 4% to about 6% natural caffeine, or about 5.4% natural caffeine. The value of the natural caffeine in the composition can be equal to an integer value or values within any of the above-described numerical ranges, including the end-points of the range. In other examples, caffeine is not included as an active composition, but other anti-anxiety and/or anti-stress active compositions are included.

Another active composition can include ashwagandha root extract and in some examples the composition contains about 2% to about 5% ashwagandha root extract, or about 3% to about 4% ashwagandha root extract, or about 3.2% ashwagandha root extract. The value of the ashwagandha root extract in the composition can be equal to an integer value or values within any of the above-described numerical ranges, including the end-points of the range.

Another active composition can include bacopa leaf extract and in some examples the composition contains about 1% to about 4% bacopa leaf extract, or about 2% to about 3% bacopa leaf extract, or about 2.7% bacopa leaf extract. The value of the bacopa leaf extract in the composition can be equal to an integer value or values within any of the above-described numerical ranges, including the end-points of the range.

The composition can contain about 0.2% to about 1% of terpenes, and more specifically, for compositions with active composition(s) that is/are known nootropic, anti-anxiety, and/or anti-stress compounds, the terpene(s) can have a complementary or similar effect. The terpene blend can contain, for example, alpha pinene, limonene, linalool, alpha-terpineol, and/or beta caryophyllene. The composition can contain, for example, about 0.3% to about 0.6% of a terpene blend, or about 0.4% to about 0.5% of a terpene blend, or about 0.41% of a terpene blend of alpha pinene, limonene, linalool, alpha-terpineol, beta caryophyllene. The value of the terpene blend in the composition can be equal to an integer value or values within any of the above-described numerical ranges, including the end-points of the range.

The composition can contain about 0.05% to 0.15% of a preservative, or about 0.1% of a preservative. The value of the preservative in the composition can be equal to an integer value or values within any of the above-described numerical ranges, including the end-points of the range.

In an exemplary formulation (Table 2: Create Composition), the formulation is comprised of about 53.8% by weight of water, about 15.4% by weight of flavoring, about 8% by weight of emulsifier, about 7% by weight of stevia leaf extract, about 5.4% by weight of caffeine (natural or artificial), about 4% by weight of glycerin, about 3.2% by weight of ashwagandha root extract, about 2.7% by weight of bacopa leaf extract, about 0.41% by weight of a terpene blend (alpha pinene, limonene, linalool, alpha-terpineol, beta caryophyllene), and about 0.1% by weight of a preservative.

TABLE 2
Create Composition

| | | | | Quantitative (by | |
|---|---|---|---|---|---|
| Qualitative | | | | Weight (KG, G, | |
| Description | | | | MG, MCG) or | |
| (Common | | | Quantitative | Volume (KL, L, | |
| Name): | Source Name: | Function: | (%): | ML): | |
| Purified Water | Aqua | Water | 53.8% | 538 | mg |
| Natural Flavors | Fruit extracts, | Flavor | 15.4% | 133 | mg |
| | Citric acid | | | | |
| Lecithin | Sunflower | Excipient - | 8.0% | 80 | mg |
| | | Emulsifier | | | |
| Stevia Leaf | Stevia | Sweetener | 7.0% | 70 | mg |
| Extract | rebaudiana | | | | |
| Natural Caffeine | Green Tea leaf | Active- | 5.4% | 54 | mg |
| | extract (Camellia | nootropic | | | |
| | sinensis) | | | | |
| Glycerin | Palm kernel | Sweetener | 4.0% | 40 | mg |
| | (Elaeis | | | | |
| | guineensis) | | | | |
| Ashwagandha | Withania | Active- anti- | 3.2% | 32 | mg |
| root extract | somnifera | anxiety, anti- | | | |
| | | stress | | | |
| Bacopa leaf | Bacopa | Active- anti- | 2.7% | 27 | mg |
| extract | monniera | anxiety, anti- | | | |
| | | stress | | | |
| Terpenes- alpha | Pine oil | Nootropic, | 0.41% | 4.1 | mg |
| pinene, | (Cupressus | relaxant, | | | |
| limonene, | macrocarpa), | entourage effect | | | |
| linalool, alpha- | Lemon (Limon | | | | |
| terpineol, beta | citrus), Lavender | | | | |
| caryophyllene | (Lavendula | | | | |
| | angustifolia), | | | | |
| | Black pepper | | | | |
| | (Piper nigrum) | | | | |
| Potassium | Synthesis | Preservative | 0.1% | 1 | mg |

Sorbate

In another example, the composition can contain from about 50% to about 75% by weight of water or another hydrophilic liquid, about 4% to about 12% by weight of natural flavors, about 6% to about 12% by weight of emulsifier, about 5% to about 15% by weight of sweetener(s), about 5% to about 15% of one or more active compositions, about 0.2% to about 1% of terpenes, and about 0.05% to 0.15% of a preservative.

More specifically, the composition can contain from about 58% to about 75% water, or about 63% to about 70% water, or about 67.5% water. The value of the water in the composition can be equal to an integer value or values within any of the above-described numerical ranges, including the end-points of the range.

The composition can contain from about 4% to about 12% by weight natural flavoring (such as fruit extracts, citric acid, etc.), or about 6% to about 10% by weight of natural flavoring, or about 8% by weight natural flavoring. The value of the natural flavoring in the composition can be equal to an integer value or values within any of the above-described numerical ranges, including the end-points of the range.

The composition can contain about 5% to about 10% by weight of emulsifier, or about 6% to about 12% by weight of emulsifier, or about 8% to about 10% by weight of emulsifier, or about 9% by weight of emulsifier. The value of the natural flavoring in the composition can be equal to an integer value or values within any of the above-described numerical ranges, including the end-points of the range.

The composition can contain about 2% to about 12% by weight of sweetener, or about 4% to about 10% by weight of sweetener, or about 7% by weight of sweetener. The value of the sweetener in the composition can be equal to an integer value or values within any of the above-described numerical ranges, including the end-points of the range. Additionally, more than one sweetener can be used to achieve this overall amount of sweetener within the composition. For example, the composition can contain about 0.5% to about 4.5% by weight of stevia leaf extract, or about 1.5% to about 3.5% by weight of stevia leaf extract, or about 2.5% by weight of stevia leaf extract. The value of the stevia leaf extract in the composition can be equal to an integer value or values within any of the above-described numerical ranges, including the end-points of the range. The composition can also contain about 2% to about 6% by weight of glycerin, or about 3% to about 5% by weight of glycerin, or about 4.5% by weight of glycerin. The value of the glycerin in the composition can be equal to an integer value or values within any of the above-described numerical ranges, including the end-points of the range. The use of glycerin as a sweetener can also have the added benefit of stabilizing the emulsifier to improve liposomal deliver of active compositions.

The composition can contain about 5% to about 15% of one or more active compositions, such as one or more active compositions known to be anti-anxiety, and/or anti-stress. One exemplary active composition can include magnolia bark and in some examples the composition contains about 3% to about 7% magnolia bark, or about 4% to about 6% magnolia bark, or about 5% magnolia bark. The value of the magnolia bark in the composition can be equal to an integer value or values within any of the above-described numerical ranges, including the end-points of the range. Another active composition can be the known relaxant passionflower extract and in some examples the composition contains about 0.5% to about 4% passionflower extract, or about 1% to about 3% passionflower extract, or about 2% passionflower extract. The value of the passionflower extract in the composition can be equal to an integer value or values within any of the above-described numerical ranges, including the end-points of the range. Another active composition can be the known relaxant chamomile flower extract and in some examples, the composition contains about 0.5% to about 1.5% passionflower extract, or about 1% passionflower extract. The value of the chamomile flower extract in the composition can be equal to an integer value or values within any of the above-described numerical ranges, including the end-points of the range.

The composition can contain about 0.2% to about 1% of terpenes, and more specifically, for compositions with active composition(s) that is/are known relaxants, anti-anxiety, entourage effect anti-anxiety, and/or anti-stress compounds, the terpene(s) can have a complementary or similar effect. The terpene blend can contain, for example, limonene, terpinolene, linalool, alpha-terpineol, and/or beta caryophyllene. The composition can contain, for example, about 0.3% to about 0.6% of a terpene blend, or about 0.4% to about 0.5% of a terpene blend, or about 0.41% of a terpene blend including limonene, terpinolene, linalool, alpha-terpineol, beta caryophyllene. The value of the terpene blend in the composition can be equal to an integer value or values within any of the above-described numerical ranges, including the end-points of the range.

The composition can contain about 0.05% to 0.15% of a preservative, or about 0.1% of a preservative. The value of the preservative in the composition can be equal to an integer value or values within any of the above-described numerical ranges, including the end-points of the range.

In an exemplary formulation (Table 3: Slumber Composition), the formulation is comprised of about 67.5% by weight of water, about 8% by weight of flavoring, about 9% by weight of emulsifier, about 2.5% by weight of stevia leaf extract, about 4.5% by weight of glycerin, about 5% by weight of magnolia bark extract, about 2% by weight of passionflower extract, about 1% by weight of chamomile flower extract, about 0.41% by weight of a terpene blend (limonene, terpinolene, linalool, alpha-terpineol, beta caryophyllene), and about 0.10% by weight of a preservative.

TABLE 3
Slumber Composition

| | | | | Quantitative (by | |
|---|---|---|---|---|---|
| Qualitative | | | | Weight (KG, G, | |
| Description | | | | MG, MCG) or | |
| (Common | | | Quantitative | Volume (KL, L, | |
| Name): | Source Name: | Function: | (%): | ML): | |
| Purified Water | Aqua | Water | 67.49% | 674.9 | mg |
| Lecithin | Sunflower | Excipient - | 9.0% | 90 | mg |
| | | Emulsifier | | | |
| Natural Flavors | Fruit extracts, | Flavors | 8.0% | 80 | mg |
| | Citric acid | | | | |
| Magnolia Bark | Magnolia | Active, relaxant | 5.0% | 50 | mg |
| Extract | officiinalis | | | | |
| Glycerin | Palm kernel | Sweetener | 4.5% | 45 | mg |
| | (Elaeis | | | | |
| | guineensis) | | | | |
| Stevia Leaf | Stevia | Sweetener | 2.5% | 25 | mg |
| Extract | rebaudiana | | | | |
| Passionflower | Passiflora | Active, relaxant | 2.0% | 20 | mg |
| Extract | incarnata | | | | |
| Chamomile | Matricaria | Active, relaxant | 1.0% | 10 | mg |
| Flower Extract | chamomile | | | | |
| Terpenes- | Lemon (limon | Relaxant, Anti- | 0.41% | 4.1 | mg |
| limonene, | citrus), Tea tree | Anxiety, | | | |
| terpinolene, | (Mellaleuca | entourage effect | | | |
| linalool, alpha- | alternifolia), | | | | |
| terpineol, beta | Lavender | | | | |
| caryophyllene | (Lavendula | | | | |
| | angustifolia), | | | | |
| | Pine oil | | | | |
| | (Cupressus | | | | |
| | macrocarpa), | | | | |
| | Black pepper | | | | |
| | (Piper nigrum) | | | | |
| Potassium | Synthesis | Preservative | 0.1% | 1 | mg |

Sorbate

In another example, the composition can contain from about 50% to about 75% by weight of water or another hydrophilic liquid, about 4% to about 12% by weight of natural flavors, about 6% to about 12% by weight of emulsifier, about 5% to about 15% by weight of sweetener(s), about 5% to about 15% of one or more active compositions, about 0.2% to about 1% of terpenes, and about 0.05% to 0.15% of a preservative.

More specifically, the composition can contain from about 58% to about 75% water, or about 63% to about 70% water, or about 67.5% water. The value of the water in the composition can be equal to an integer value or values within any of the above-described numerical ranges, including the end-points of the range.

The composition can contain from about 4% to about 12% by weight natural flavoring (such as fruit extracts, citric acid, etc.), or about 6% to about 10% by weight of natural flavoring, or about 8% by weight natural flavoring. The value of the natural flavoring in the composition can be equal to an integer value or values within any of the above-described numerical ranges, including the end-points of the range.

The composition can contain about 5% to about 10% by weight of emulsifier, or about 6% to about 12% by weight of emulsifier, or about 8% to about 10% by weight of emulsifier, or about 9% by weight of emulsifier. The value of the natural flavoring in the composition can be equal to an integer value or values within any of the above-described numerical ranges, including the end-points of the range.

The composition can contain about 2% to about 12% by weight of sweetener, or about 4% to about 10% by weight of sweetener, or about 6.5% by weight of sweetener. The value of the sweetener in the composition can be equal to an integer value or values within any of the above-described numerical ranges, including the end-points of the range. Additionally, more than one sweetener can be used to achieve this overall amount of sweetener within the composition. For example, the composition can contain about 0.5% to about 4.5% by weight of stevia leaf extract, or about 1.5% to about 3.5% by weight of stevia leaf extract, or about 2.5% by weight of stevia leaf extract. The value of the stevia leaf extract in the composition can be equal to an integer value or values within any of the above-described numerical ranges, including the end-points of the range. The composition can also contain about 2% to about 6% by weight of glycerin, or about 3% to about 5% by weight of glycerin, or about 4.5% by weight of glycerin. The value of the glycerin in the composition can be equal to an integer value or values within any of the above-described numerical ranges, including the end-points of the range. The use of glycerin as a sweetener can also have the added benefit of stabilizing the emulsifier to improve liposomal deliver of active compositions.

The composition can contain about 5% to about 15% of one or more active compositions, such as one or more active compositions known to be relaxant(s). One exemplary active composition/supplement can include GABA and in some examples the composition contains about 3% to about 7% GABA, or about 4% to about 5% magnolia bark, or about 4.3% GABA. The value of the GABA in the composition can be equal to an integer value or values within any of the above-described numerical ranges, including the end-points of the range. Another active composition can be the known relaxant rhodiola root and in some examples the composition contains about 1% to about 6% passionflower extract, or about 3% to about 4% passionflower extract, or about 3.2% passionflower extract. The value of the passionflower extract in the composition can be equal to an integer value or values within any of the above-described numerical ranges, including the end-points of the range.

The composition can contain about 0.3% to about 1% of terpenes, and more specifically, for compositions with active composition(s) that is/are known relaxants, the terpene(s) can have a complementary or similar effect. The terpene blend can contain, for example beta caryophyllene, linalool, alpha-terpineol, and/or limonene. The composition can contain, for example, about 0.4% to about 0.9% of a terpene blend, or about 0.6% to about 0.7% of a terpene blend, or about 0.61% of a terpene blend including limonene, terpinolene, linalool, alpha-terpineol, beta caryophyllene. The value of the terpene blend in the composition can be equal to an integer value or values within any of the above-described numerical ranges, including the end-points of the range.

The composition can contain about 0.05% to 0.15% of a preservative, or about 0.1% of a preservative. The value of the preservative in the composition can be equal to an integer value or values within any of the above-described numerical ranges, including the end-points of the range.

In an exemplary formulation (Table 4: Chill Composition), the formulation is comprised of about 69% by weight of water, about 6.2% by weight of flavoring, about 9% by weight of emulsifier, about 2% by weight of stevia leaf extract, about 4.5% by weight of glycerin, about 4.3% by weight of GABA, about 3.2% by weight of rhodiola root, about 1.1% by weight of L-Theanine, about 0.61% by weight of a terpene blend (beta caryophyllene, linalool, alpha-terpineol, limonene), and about 0.1% by weight of a preservative.

TABLE 4
Chill composition

| | | | | Quantitative (by | |
|---|---|---|---|---|---|
| Qualitative | | | | Weight (KG, G, | |
| Description | | | | MG, MCG) or | |
| (Common | | | Quantitative | Volume (KL, L, | |
| Name): | Source Name: | Function: | (%): | ML): | |
| Purified Water | Aqua | Water | 68.9% | 689 | mg |
| Lecithin | Sunflower | Excipient - | 9.0% | 90 | mg |
| | | Emulsifier | | | |
| Natural Flavors | Fruit extracts, | Flavors | 6.2% | 62 | mg |
| | Citric acid | | | | |
| Glycerin | Palm kernel | Sweetener | 4.5% | 45 | mg |
| | (Elaeis | | | | |
| | guineensis) | | | | |
| GABA | Gamma | Active- relaxant | 4.3% | 43 | mg |
| | aminobutyric | | | | |
| | acid (synthesis) | | | | |
| Rhodiola root | Rhodiola rosea | Active- relaxant | 3.2% | 32 | mg |
| Stevia Leaf | Stevia | Sweetener | 2.0% | 20 | mg |
| Extract | rebaudiana | | | | |
| L-Theanine | synthesis | Active, relaxant | 1.1% | 11 | mg |
| Terpenes- beta | Black pepper | Relaxant, Anti- | 0.61% | 6.1 | mg |
| caryophyllene, | (Piper nigrum), | Anxiety, | | | |
| linalool, alpha- | Lavender | entourage effect | | | |
| terpineol, | (Lavendula | | | | |
| limonene | angustifolia), | | | | |
| | Pine oil | | | | |
| | (Cupressus | | | | |
| | macrocarpa), | | | | |
| | Lemon (limon | | | | |
| | citrus) | | | | |
| Potassium | Synthesis | Preservative | 0.1% | 1 | mg |

Sorbate

Reference in the specification to “one configuration” ˜ “one embodiment,” “one formulation,” “a configuration” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” in various places may not necessarily limit the inclusion of a particular element of the invention to a single configuration, rather the element may be included in other or all configurations discussed herein.

As used herein, the term “about” is used to provide flexibility to a numerical range endpoint by providing that a given value may be “a little above” or “a little below” the endpoint while still accomplishing the function associated with the range, for example, “about” can encompass variations of 20%, ±10%, ±5%, +1%, ±0.5%, or even ±0.1% of the specified amount. As used herein, a plurality of items, structural elements, compositional elements, and/or materials may be presented in a common list for convenience. However, these lists should be construed as though each member of the list is individually identified as a separate and unique member.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 5 to about 60” should be interpreted to include not only the explicitly recited values of about 1 to about 5, but also include individual values and sub-ranges within the indicated range. Thus, included in this numerical range are individual values such as 6, 7, 8, 9, etc., through 60, and sub-ranges such as from 10-20, from 30-40, and from 50-60, etc., as well as each number individually. This same principle applies to ranges reciting only one numerical value as a minimum or a maximum. Furthermore, such an interpretation should apply regardless of the breadth of the range or the characteristics being described.

Any formulations described herein are intended to encompass formulations which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined.

Although the foregoing disclosure provides many specifics, such as use of the formulation for transdermal delivery, it will be appreciated other applications, such as use for topical delivery, are contemplated and these should not be construed as limiting the scope of any of the ensuing claims. Other embodiments and configurations may be devised which do not depart from the scopes of the claims. Features from different embodiments and configurations may be employed separately or in combination. Accordingly, all additions, deletions and modifications to the disclosed subject matter that fall within the scopes of the claims are to be embraced thereby. The scope of each claim is indicated and limited only by its plain language and the full scope of available legal equivalents to its elements.

Furthermore, if any references have been made to patents and printed publications throughout this disclosure, each of these references and printed publications are individually incorporated herein by reference in their entirety.

1. A composition for oral mucosal delivery comprising:
from 60% to 70% by weight water;
from 10% to 20% by weight flavoring;
from 5% to 10% by weight emulsifier;
from 3% to 9% by weight sweetener;
from 0.2% to 1% by weight a terpene blend, the terpene blend comprising at least two terpenes having anti-inflammatory properties selected from the group consisting of terpinolene, beta caryophyllene, alpha pinene, linalool, alpha-terpineol, limonene, and combinations thereof;
from 3% to 6% by weight of a supplement having anti-inflammatory properties, the supplement selected from the group of cat's claw root extract, boswellia root extract, and combinations thereof.

2. The composition of claim 1, wherein the flavoring comprises citric acid.

3. The composition of claim 2, wherein the composition comprises from 13% to 14% by weight citric acid.

4. The composition of claim 1, wherein the emulsifier comprises 8% by weight lecithin.

5. The composition of claim 1, wherein the supplement comprises 3% to 3.5% by weight cat's claw root extract and 2.5% to 3% by weight of boswellia root extract.

6. The composition of claim 1, wherein the terpene blend comprises 0.6% to 0.7% by weight of a blend of terpinolene, beta caryophyllene, alpha pinene, linalool, alpha-terpineol, and limonene.

7. The composition of claim 1, wherein the sweetener comprises a blend of 3% to 5% glycerin and 1% to 3% by weight stevia leaf extract.

8. A composition for oral mucosal delivery comprising:
at least two terpenes having anti-inflammatory properties, the at least two terpenes comprising at least one of terpinolene, beta caryophyllene, alpha pinene, linalool, alpha-terpineol, limonene; and
at least two supplements having anti-inflammatory properties, the supplements comprising at least one of cat's claw root extract and boswellia root extract.

9. A composition for oral mucosal delivery comprising:
from 50% to 60% by weight water;
from 10% to 20% by weight flavoring;
from 5% to 10% by weight emulsifier;
from 7% to 15% by weight sweetener;
from 0.2% to 1% by weight a terpene blend, the terpene blend comprising at least two terpenes having anti-anxiety properties selected from the group consisting of alpha pinene, limonene, linalool, alpha-terpineol, beta caryophyllene, and combinations thereof,
from 6% to 12% by weight of a supplement having anti-anxiety properties, the supplement selected from the group of ashwagandha root extract, bacopa leaf extract, caffeine, and combinations thereof.

10. The composition of claim 9, wherein the emulsifier aids liposomal delivery of the supplement, and wherein the emulsifier comprises 8% by weight lecithin.

11. The composition of claim 9, wherein the supplement comprises 5% to 6% by weight caffeine, 3% to 3.5% by weight ashwagandha root extract, and 2.5% to 3% by weight of bacopa leaf extract.

12. The composition of claim 9, wherein the terpene blend comprises 0.6% to 0.7% by weight of a blend of terpinolene, beta caryophyllene, alpha pinene, linalool, alpha-terpineol, and limonene.

13. The composition of claim 9, wherein the sweetener comprises a blend of 3% to 5% glycerin and 1% to 3% by weight stevia leaf extract.

14. A composition for oral mucosal delivery comprising:
from 60% to 70% by weight water;
from 10% to 20% by weight flavoring;
from 6% to 11% by weight emulsifier;
from 5% to 10% by weight sweetener;
from 0.2% to 1% by weight a terpene blend, the terpene blend comprising at least two terpenes having anti-anxiety properties selected from the group consisting of limonene, terpinolene, linalool, alpha-terpineol, beta caryophyllene and combinations thereof;
from 5% to 10% by weight of a supplement having anti-anxiety properties, the supplement selected from the group of magnolia bark extract, passionflower extract, chamomile flower extract, and combinations thereof.

15. The composition of claim 14, wherein the supplement comprises 4% to 6% by weight magnolia bark extract, 1% to 3% by weight passionflower extract, and 0.5% to 1.5% by weight of chamomile flower extract.

16. The composition of claim 14, wherein the terpene blend comprises 0.6% to 0.7% by weight of a blend of limonene, terpinolene, linalool, alpha-terpineol, and beta caryophyllene.

17. The composition of claim 14, wherein the sweetener comprises a blend of 3% to 5% glycerin and 1% to 3% by weight stevia leaf extract.

18. A composition for oral mucosal delivery comprising:
from 65% to 75% by weight water;
from 1% to 10% by weight flavoring;
from 6% to 12% by weight emulsifier;
from 5% to 10% by weight sweetener;
from 0.2% to 1% by weight a terpene blend, the terpene blend comprising at least two terpenes having anti-anxiety properties selected from the group consisting of beta caryophyllene, linalool, alpha-terpineol, limonene, and combinations thereof,
from 5% to 10% by weight of a supplement having anti-anxiety properties, the supplement selected from the group of GABA, rhodiola root, L-Theanine, and combinations thereof.

19. The composition of claim 20, wherein the supplement comprises 4% to 5% by weight GABA, 3% to 4% by weight Rhodiola rosea, and 0.5% to 1.5% by weight L-theanine.

20. The composition of claim 20, wherein the terpene blend comprises 0.5% to 0.7% by weight of a blend of beta caryophyllene, linalool, alpha-terpineol, and limonene.
